(19) United States
(12) Patent Application Publication
(10) Pub. No.: US 2007/0208248 A1
(43) Pub. Date: Sep. 6, 2007

(54) NON-EXPERT CONTROL OF AN MR SYSTEM

(75) Inventors: Paul Royston Harvey, Eindhoven (NL); Hans Hermanus Tuithof, Eindhoven (NL); Sander Slegt, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 10/599,141

(22) PCT Filed: Mar. 15, 2005
(86) PCT No.: PCT/IB05/50906

§ 371 Date: Sep. 21, 2006

(30) Foreign Application Priority Data
Mar. 26, 2004 (EP) 04101269.1

(51) Int. Cl. A61B 5/055

(52) U.S. Cl. 600/410

(57) ABSTRACT

The invention relates to magnetic resonance imaging and offers a way to provide even the non expert user with full control of a magnetic resonance imager. The invention achieves this by offering a user interface in is arranged to assign values to at least one attribute used to influence the visual presentation of an acquired magnetic resonance image and in which the values of the at least one attribute are arranged to be chosen from information indicating the effects of their assignment on the content of the visually presented acquired magnetic resonance image.

The invention relates to a user interface for a magnetic resonance imager, arranged to assign values to at least one attribute used to influence the visual presentation of an acquired magnetic resonance image.

U.S. Pat. No. 6,400,157 discloses a magnetic resonance system for the remote control of a magnetic resonance imager. Specifically, it describes a system for the control of such an imager by a surgeon utilizing magnetic resonance scanning during a surgical procedure. Such scanning may be useful, for example, for the localization of surgical tools during neurosurgery. The surgeon has control of a computer, for example, which allows him to control scan parameters such as echo time TE, repetition time TR, slice axis and resolution.

The system described in U.S. Pat. No. 6,400,157 requires the user, in this case a surgeon, to manipulate the same scan parameters that would normally be under the control of the technologist. The surgeon therefore needs detailed knowledge of the physical production of a magnetic resonance image in order to correctly control these parameters. Even assuming the surgeon in this case has acquired considerable knowledge of magnetic resonance scanning, the system disclosed in U.S. Pat. No. 6,400,157 nevertheless transfers control of the scan from the specialist in magnetic resonance scan acquisition, to someone who is by their training an expert in something other than the technique of magnetic resonance scanning.

It is an object of the invention to produce a user interface which can be used by someone who is not an expert in magnetic resonance imaging. This is achieved according to the method of the invention by which the values of the at least one attribute are arranged to be chosen from information indicating the effects of their assignment on the content of the visually presented acquired magnetic resonance image.

Magnetic resonance scanning of a human body requires the interaction between superpositions of externally applied magnetic fields, radiofrequency fields and the nuclear magnetic moments of the nuclei making up the human body and is considered to be a complicated imaging modality to understand. It is agreed among magnetic resonance professionals that correct and advantageous operation of a magnetic resonance imager requires training and detailed knowledge of the interaction between a large number of variables. Without this knowledge the acquisition of magnetic resonance scans can become a chance affair relying on the experimental manipulation of variables into combinations which may or may not produce a diagnostically useful scan.

Magnetic resonance imaging produces medical images with an advantageous differentiation between many soft tissue types at a resolution which renders it diagnostically useful. This makes magnetic resonance imaging attractive in comparison to conventional X-ray imaging modalities, such as X-ray computed tomography, which do not show good soft tissue contrast. There has consequently been a move in radiology to extend the application of magnetic resonance into increasingly greater numbers of clinical areas. Some of these areas involve participation by medical personnel, not all of whom are experts in magnetic resonance imaging techniques.

One such example is the extension of magnetic resonance into interventional surgery. Here magnetic resonance imaging is used during surgical procedures to enable the surgeon to image and consequently visualize the anatomy on which he or she is operating. The patient and surgeon must be physically present at the site of a suitable imager and the images acquired must be made available to the surgeon in such a way that they are visible from the position in which he or she is operating. In order for the surgeon to gain maximum benefit from the availability of magnetic resonance imaging under these circumstances, the surgeon should be able to control the imaging process directly. This can easily be explained if one considers that it is the surgeon in the process of making an incision, and therefore requiring very specific and exact visual information about the anatomy surrounding the site of that incision, who is best placed to assess whether the image presented to him does indeed show the anatomical area under consideration. Similarly, if the image presented does not show the exact anatomy required in a suitable manner, the surgeon is the person best placed to consider by how much the image plane should be altered before it aligns with the position of the surgical knife and further, if the image is not sufficiently clear, in what way the image contrast or resolution should be changed. There are therefore good reasons why the control of the imaging device should be handed over to the surgeon.

However, the surgeon, although highly expert in his own field, is rarely an expert in magnetic resonance imaging. The invention allows such a non-expert user to correctly control the acquisition of the image by allowing the user to control the presentation of the magnetic resonance image using a number of attributes, each of which has an effect on the resulting magnetic resonance image.

The different pieces of equipment in a magnetic resonance imager are controlled by input values which govern such image qualifiers as, say, spin sequence selection, slice or volume selection, slice thickness or alignment of slices. These input values are translated into various scan parameters by the control system of the imager. The scan parameters directly control the operation of the individual pieces of equipment such as the coils producing the gradient fields or the transmit or receive radiofrequency coil. The invention alters the way in which the information governing the choice of scan parameters is passed from the user to the control system of the imager. In normal use the input values would be entered by the user into an input screen on a control console and would be passed on as a series of instructions to the system control. So for example, values directing the precise working of the gradient coils can be provided for the control system by inputting the precise plane alignment of the required images. A requirement for a particular plane alignment of the resultant slice images therefore becomes a combination of varying gradient field strengths which produces magnetic resonance signal originating in geometrical planes of that precisely required alignment.

In a similar way, the selection of spin sequence provides the control system with instructions to precisely time the relative workings of the transmit radiofrequency coil with the various gradient coils and the receive radiofrequency coil. This elicits magnetic resonance signals from the subject which when reconstructed into an image will show tissue contrast of varying degrees and types, depending on the spin sequence selected.

Further, selection by the user of various additional modes of operation for the magnetic resonance imager instructs the control system how to formally acquire the data which is generated by the interplay of magnetic and radiofrequency fields. For example, the imager, via the control system, can be instructed to acquire the data in any one of several data orders, each with their own advantages and disadvantages. An example of this would be the choice of which sampling of k-space to use. The order and rate in which data is sampled in k-space impinges directly on the resultant image quality. Images involving anatomical movement, such as the beating of the heart or the flow of paramagnetic contrast media through the arterial system, benefit from a sampling order which acquires data from the center of k-space before data from the periphery. This increases the proportion of image signal which encodes gross anatomical detail at the expense of image signal which encodes fine detail, but produces a diagnostically useful image when there is limited time available.

Various sampling orders have been developed to achieve this acquisition of magnetic resonance data and the choice between them can be made by the experienced operator and inputted into the screen of the control console.

In addition to this, the imager can be instructed to sample a high density of data using the radiofrequency receive coil or instructed to sample a subset of data and to reconstruct the missing data from the sample subset. For example, it is possible to sample only half or even a quarter of the data from high frequency k-space and reconstruct the remaining unsampled data from the conjugate of the sampled data. Again, the choice of whether or not to do this within the context of a particular magnetic resonance imaging acquisition is best made by an experienced operator.

The entering of values into the input screen of a control console may be carried out in any one of several known ways including the use of a cursor with keyboard or mouse control and by use of a joystick. The entering of data frequently follows a set pattern defined by system menus presented to the user during scan sessions. The system menu would allow, for example, the user to control the input values for the position of the scan planes, the orientation of the scan planes, the size of the scan planes and the spin sequence used and the resolution of the final scan. The data can be entered in any order that the system menu allows.

Instead of inputting the normal data values used to control the scan parameters, the invention allows the user to provide information to the control system of the magnetic resonance imager which is defined in terms of the result it produces. In other words, rather than the user have to know and understand what the effects of a particular scan parameter or input value are, he chooses the result he would like to have and inputs this as the information to the control system. The control system then interprets these instructions into the necessary scan parameters. In other words, the user chooses certain attributes, the choice of which has a direct effect on the final presentation of the magnetic resonance scan which the user is directing.

The information defining the results of the scan can be offered to the user in various ways.

To avoid confusion on the part of the non expert user, the information is offered in the form of discrete choices. In the example of the surgeon as non expert user, the user is already faced with a multitude of choices and visual spatial tasks at the point in time at which he is using and controlling the magnetic resonance imager. Offering such a user only a discrete number of choices limits the amount of information to which he is exposed and to which he is forced to respond. The information can be offered as a discrete list of options and such a list can be a set of descriptions of the result of each choice. So for example, a surgeon performing a surgical procedure on the cranium and wishing to perform a scan of the part of the brain on which he is operating may be confronted with a list of two possible options for spin sequence, one of which is described as ‘white matter enhanced scan’ and the other of which is entitled ‘grey matter enhanced scan’. Similarly, the various spin sequences as they are applied to other areas of gross anatomy can be similarly described in terms of their most noticeable visual differentiating feature.

It has been found that one particularly successful presentation is a visual presentation. This allows the user to assess the choice of attributes in the same way in which he would assess the resultant image and in doing so this reduces the overall number of different forms of information vying for the user's attention. In the case of a visual presentation, the choice of, say, the attribute of spin sequence is offered to the user in the form of a series of discrete representative images, each one offering a sample of how the image would look if acquired using that particular spin sequence. The user chooses from the discrete list or array of visual sample images the one which most looks like the form of resultant image he would like to have. That choice can be inputted into the system as an input attribute and is then translated by the control system into the required parameters needed to apply the chosen spin sequence to the magnetic resonance imager.

A particularly useful way of allowing the user to input the values is through voice control. In the case of user control by a surgeon this allows direct control of the system without any physical contact between the user and the control console, thus avoiding either contamination by the user or of the user compromising their sterile status.

Voice control can be accomplished by normal methods of voice control, for example by microphone and voice recognition software. The user interface may be constructed in such a way that the input values, while inputted to the system control via a voice control system, are nevertheless reproduced visually on the input screen so that the user can keep track of what instructions have been already inputted to the magnetic resonance imager.

In order to facilitate control of the system and selection of the attributes, a set of voice commands can be formulated which are used to control the system. This set of voice commands is rationalized to a minimum set of commands which are not specifically dependent on a deep or in-depth knowledge of magnetic resonance imaging. This allows the user to control the magnetic resonance system without any knowledge of the terms used in magnetic resonance imaging. The selection of attributes at the input screen is therefore made using ordinary words and commands, these words and commands sometimes being strung together to form an overall sequences of commands.

These and other aspects of the invention will be further explained with the help of the following figures.

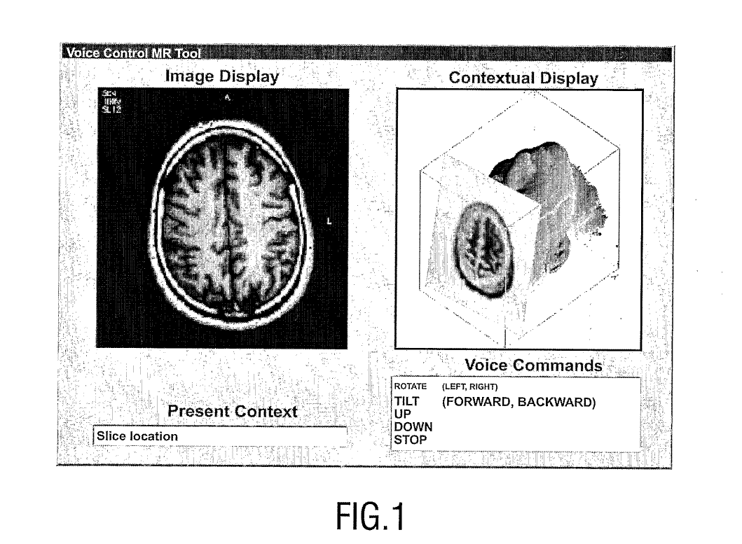

FIG. 1 shows a dual display system which can be shown on the user interface of a magnetic resonance imager using the invention, in which dual display system one display is used for the current image data and the other display is used to provided the contextual information utilized to provide the user which the choice of attribute over which the invention gives him control.

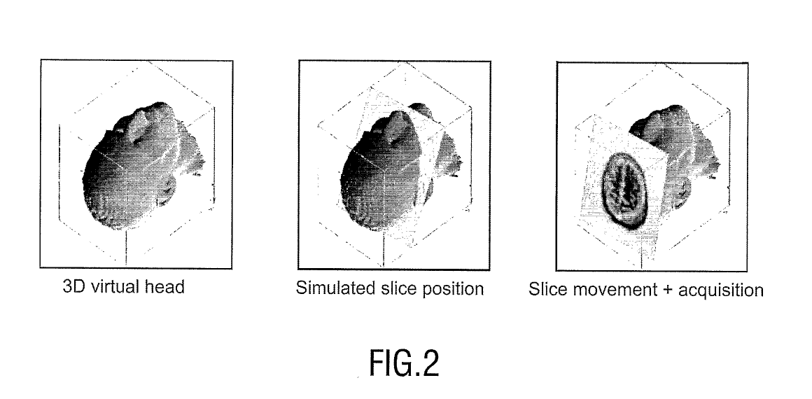

FIG. 2 shows thee movement of the scanning plane to new slice locations or orientations simulated on a separately displayed 3 dimensional perspective anatomical model of the region or anatomy of interest.

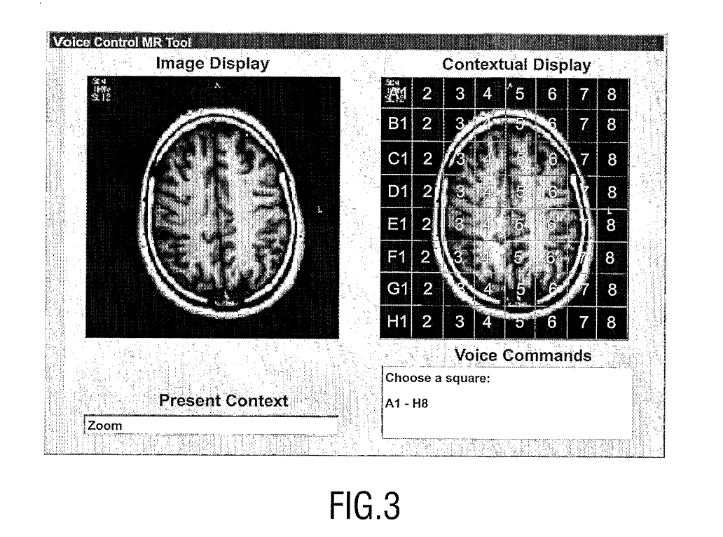

FIG. 3 shows an example of how the invention can be utilized to control the zoom in an image.

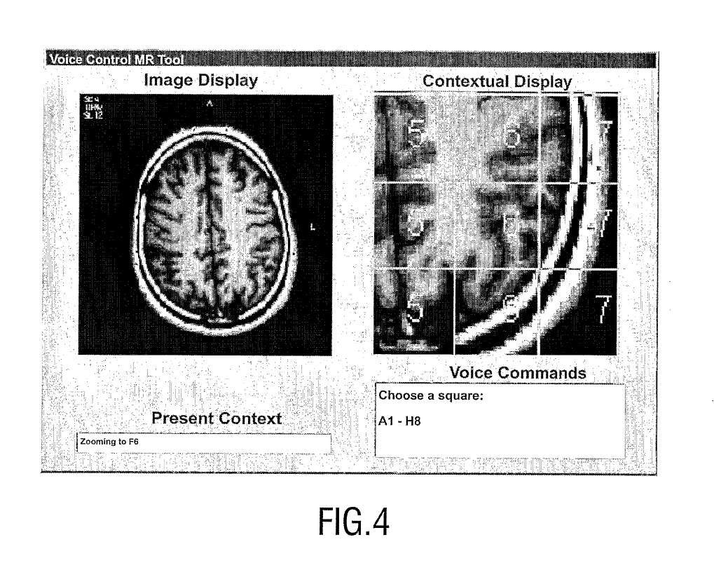

FIG. 4 shows the result of the zooming action as applied to the image in FIG. 3.

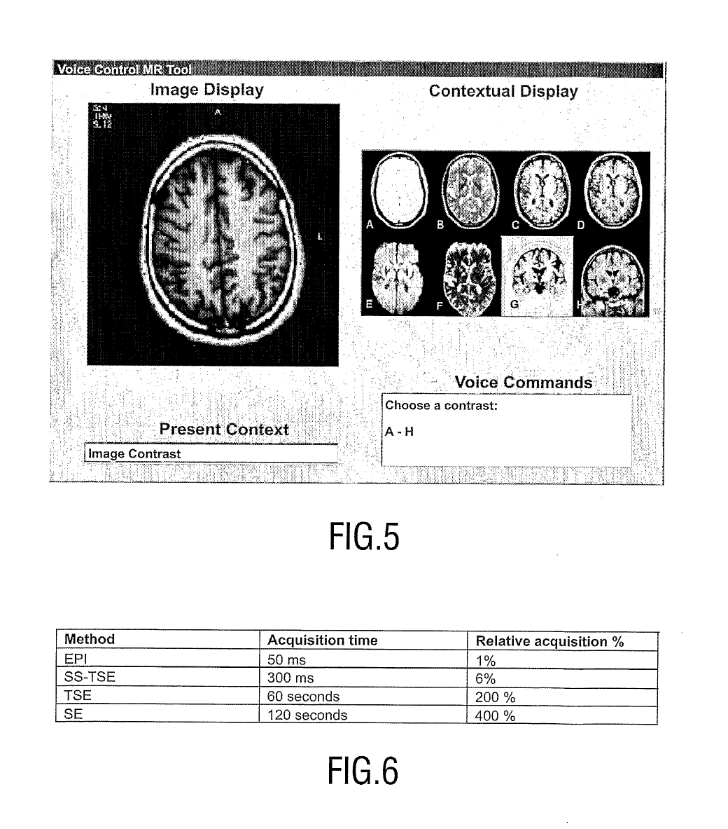

FIG. 5 shows the use of the invention to control the contrast in the image.

FIG. 6 shows a table showing the parameters used for T2 weighted imaging.

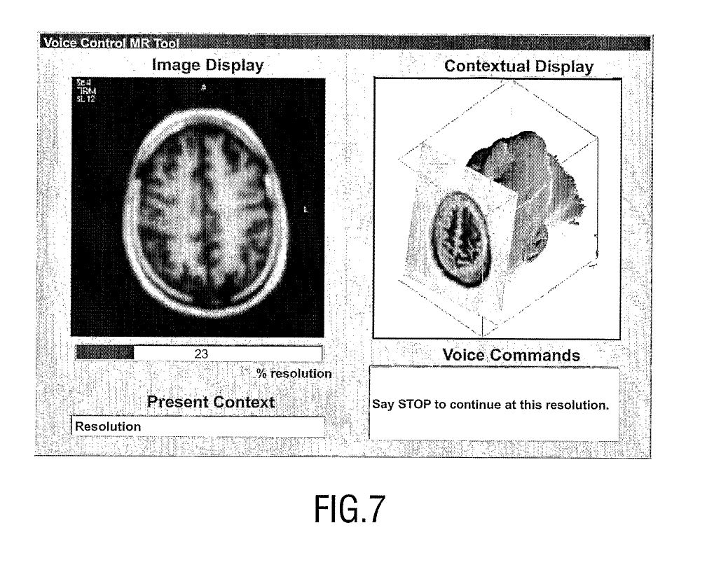

FIG. 7 shows the use of the invention to control the resolution in the image.

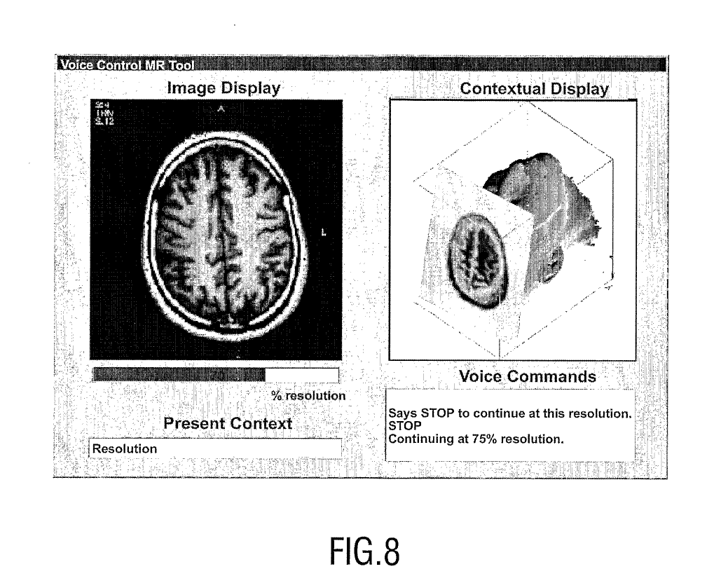

FIG. 8 shows the use of the invention to control the resolution in the image.

FIG. 1 shows a dual display system in which one display is used for the current image data and the other display is used to provided contextual information. This contextual information may be further reference images, overlays onto an existing image, 3 dimensional perspectives or indeed any further display for the purpose of providing visual cues that facilitate the user's choice of attributes. In FIG. 1 an acquired image, in this case a cranial slice, is shown on the left of the dual display system with the position of the acquired image plane shown on a contextual display, in this case a display of a head, on the right of the dual display system. This allows the user to see the context of the image just acquired and to conceptualize mentally the exact position of the slice. In a similar manner the contextual display can be used to show the position of the slice about to be acquired. In such a case the contextual display would show the contextual position of the slice already acquired, which would then alter as the user inputted more commands to control the position of the next slice to be acquired. In this way the presentation shows the user what the effect of the inputted commands would be if they were to be executed. The attributes, in this case, can loosely be described as the positional coordinates of the acquisition slice or the orientation of the acquisition slice. One may think of this orientation in terms of mathematical orthogonal coordinates which define the alignment of the image slice precisely within the anatomical space. These orthogonal coordinates could be the x, y and z co-ordinates or the r, Θ and Φ coordinates of the normal vector to the slice, for instance. These coordinates, or attributes, are chosen, in this case from the contextual display, using the result of the choice of attribute applied directly to the display itself. In other words, the display shows a virtual representation of the intended acquisition, and the user's own choice of slice orientation, or attributes, is made from this presentation.

The set of commands which allow voice control of the magnetic resonance imager can be designed to allow a simplified control of the positioning of the slice. Thus the user need not be required to stipulate the position of the intended acquisition slice in terms of geometrical coordinates. Instead, the vocabulary of the commands and the voice control system can be designed to utilize referential commands. In other words, the commands utilized to choose the attributes of alignment of the acquisition slice can signify that choice by altering the attributes which described the previous choice. As commands are issued by the user the contextual display showing the position of the acquisition slice depicts the chosen changes in the position of the acquisition slice. In other words, the position of the acquisition slice moves on the contextual display.

FIG. 2 shows this movement of the scanning plane to new slice locations or orientations. This movement of intended slice acquisition plane can also be simulated in real time, in other words while the magnetic resonance acquisition is running. It can be shown on a separately displayed 3 dimensional perspective anatomical model of the region or anatomy of interest. In this way, when the operator issues the voice command to change location, the movement of the acquisition slice, which is superimposed on the 3 dimensional model, is simulated without yet adjusting the magnetic resonance acquisition. The movement in the direction requested should be at a rate which provides adequate resolution of movement on the display therefore allowing the user to perceive when the slice is situated at a position or orientation which is clinically useful. This enables the operator to comfortably issue the command to stop the movement. When the movement is completed on the simulated slice, the magnetic resonance acquisition is updated with the new slice location information. The image of the new slice is then updated on the main image display.

The 3 dimensional perspective anatomical image of the region of interest is available in a separate contextual display with the current slice location superimposed. This is used as a visual reference for the operator just like a globe of the earth. Alternatively, a projection of the slice location, by means of laser or similar, can be made on the anatomy itself.

The sort of referential commands utilized by the system can be exemplified by, for example, the following sequence of voice commands (the commands are shown on the left, an explanation is provided on the right):
LOCATION to invoke slice location mode.
HOME move slice to a pre-defined starting position (center of anatomy).
AXIAL basic slice orientations.
CORONAL
SAGITTAL
TILT make tilt.
FORWARD start tilting simulated slice forward.
BACKWARD start tilting simulated slice backward.
STOP stop the tilting of the simulated slice and acquire at that position.
ROTATE
LEFT start rotating simulated slice left.
RIGHT
STOP stop the rotation of the simulated slice and acquire at that position.
UP move the slice position upwards along the axis perpendicular to the plane of the image.
STOP stop movement of the simulated slice and acquire at that position.
DOWN move the slice position upwards along the axis perpendicular to the plane of the image.
STOP stop movement of the simulated slice and acquire at that position.
CONTINUE finish current mode and continue acquisition at new location.

It can be seen from the above example that one way of using such a command structure is to use nested commands, so that the use of a command word allows the user access to a further menu of commands governing a particular attribute or set of attributes.

Other attributes, in addition to the orientation of the acquisition slice, can be chosen using the invention. For example, the invention can be used to alter the field of view of the acquisition or to zoom, or magnify a portion of an acquired image.

FIG. 3 shows an example of a ZOOM mode used to zoom a region of the image. As an example of how it is used the operator requests Zoom Mode. The contextual display then displays the current image with a coarse grid overlaid and each square on the grid labeled with a different index. To zoom a region, the operator chooses the grid square closest to the region of interest and utters the index which labels that square. So, for example, to zoom region F6, the user says “F6”. The chosen region is then centered in the contextual display and begins to make a digital zoom. When the level of zoom is sufficient the user complete the action by saying the word STOP. Either the acquisition is reconfigured to acquire the new field of view or the image display is interpolated to the current zoom setting. The operator can choose one or the other of these actions by saving either CONTINUE or ACQUIRE.

FIG. 4 shows the result of the zoom action.

Within a simplified command system, ordinary words are used to control the magnetic resonance imager. So for example, the command CONTINUE, following a ZOOM command, continues the current scan with the digital zoom applied to the image display. The command ACQUIRE, following a ZOOM command, cause the acquisition to be updated with the current location and zoomed field of view settings.

The invention can be used to change other attributes which can be said to describe the visual appearance of the image. An example of such an attribute could be described by the contrast in an image. As an example of how this can be achieved using the invention the user would utter a command designed to put the system into the mode of operation which allows this particular attribute to change. This could be, for example, the word CONTRAST. This would invoke a display of a number of sample or synthesized images in the contextual display which reflect a variety of different image contrasts and imaging methods.

This is shown in FIG. 5. The operator utters the label of the image or imaging method that matches the desired contrast. Having stipulated the contrast the operator would say the command CONTINUE and the system immediately switches to an imaging method that provides the requested contrast while maintaining all other attributes of the previous image which for example could be slice location and field of view.

When there are a number of different imaging methods that provide similar contrast then it is possible to provide example images from each method in order of increasing acquisition time. The acquisition time can be displayed next to the image, as a relative percentage of the whole image set, and will aid in the choice. So the operator could choose contrast C at 6% acquisition time. This would dictate a specific kind of contrast and a specific kind of imaging method. For example, a number of imaging methods are available to make a T2 weighted image. These are compared in the table shown in FIG. 6.

The invention can also be used to control image resolution. However, image resolution can also be controlled in real-time. The operator can therefore see the image resolution gradually improve with further acquisition of magnetic resonance signal data and so can choose to stop the acquisition, or fix the resolution, when the image quality is sufficient.

So for example, to invoke a change in resolution, the operator would issue the commands RESOLUTION followed by INTERACTIVE. The imaging method could then switch to a centric encoding order, while maintaining the present location and field of view, and continue with the acquisition whilst displaying the continually updated image. What the operator will see, in the image display window, is a very low resolution image that, in real time, becomes higher and higher in resolution as the higher k-space lines are sampled. The operator issues the command STOP when the desired resolution is reached. Following the STOP command, imaging continues at the chosen resolution. It is possible that the imaging method can switch back to its original encoding order if so desired.

FIGS. 7 and 8 illustrate an example of what the operator may see. The horizontal bar indicates the percentage resolution which is also related to the level of scan completion at that time. By performing the resolution choice in this way, the user not only gets a feel of the suitability of the resolution for the task at hand, but also a feel for how long it takes to reach a desired resolution. The operator can continue the scan at optimum resolution when certain desired features of the image are clear enough.

This evolving image is then always acquired in the most efficient manner that satisfies the resolution requirements of the operator.

Alternatively, following the RESOLUTION command, the operator can choose to say a fixed resolution command, “3 MM”, for example. This would simply change the imaging method to acquire at the new resolution without the interactive routine.

It can be seen that the invention offers an advantage over the use of a conventional magnetic resonance system, which even when equipped with real-time interactive magnetic resonance imaging capability, currently requires the operator to make complex parameter changes. The addition of voice control ensures even more advantages in that the use of manual input mechanisms are avoided. In the case of the surgical user this means that the surgeon's hands are still free to operate on the patient, and the surgeon can furthermore interact with the magnetic resonance imager without compromising the sterile environment encompassing the patient.

1. A user interface for a magnetic resonance imager, arranged to assign values to at least one attribute used to influence the visual presentation of an acquired magnetic resonance image, wherein the values of the at least one attribute are arranged to be chosen from information indicating the effects of their assignment on the content of the visually presented acquired magnetic resonance image.

2. A user interface as claimed in claim 1, wherein the value of the at least one attribute determines parameters which control the acquisition of magnetic resonance signal which is reconstructed to form the acquired magnetic resonance image, wherein, the values of the at least one attribute are arranged to be chosen from information indicating the effect of the determined parameters on the acquisition of the magnetic resonance signal which is reconstructed to form the acquired magnetic resonance image.

3. A user interface as claimed in claim 1, wherein, the information indicating the effects of the assignment of the attributes on the content of the visually presented magnetic resonance image is presented as a series of discrete choices.

4. A user interface as claimed in claim 3, wherein, the information presented in a series of discrete choices is presented as a series of visual samples.

5. A user interface as claimed in claim 1, wherein
it comprises a visual presentation means for presenting the effects of the assignment of the attribute to the user,
and further comprises an instruction input means to convey the assignment of the value of the at least one attribute to the magnetic resonance imager.

6. A user interface as claimed in claim 5, wherein, the value of the attribute is conveyed to the magnetic resonance imager through voice control.

7. A user interface as claimed in claim 1, wherein, the at least one attribute is arranged to be chosen from the user interface during the acquisition of magnetic resonance image.

8. A user interface as claimed in claim 7, wherein
the content of the visual presentation of the magnetic resonance image is updated via the user interface during acquisition of the magnetic resonance image,
and the at least one attribute is arranged to be chosen from the user interface during the evolution of the updateable presentation of the content of the magnetic resonance image.

9. A user interface as claimed in claim 7, wherein, the at least one attribute is image resolution.

10. A user interface as claimed in claim 9, wherein, the magnetic resonance image is acquired using a centric encoding order.

11. A computer program used to control a user interface for the acquisition of a magnetic resonance scan, the computer program being arranged to assign values to at least one attribute used to influence the visual presentation of an acquired magnetic resonance image, wherein,
the computer program is arranged to present information indicating the effects of the values on the content of the visually presented acquired magnetic resonance image,
and the computer program being further arranged to receive the values as input values.

12. A magnetic resonance system for the acquisition of magnetic resonance images, wherein the magnetic resonance system is arranged to assign values to at least one attribute used to influence the visual presentation of an acquired magnetic resonance image, wherein
the magnetic resonance system is arranged to present information indicating the effects of the values on the content of the visually presented acquired magnetic resonance image,
the magnetic resonance system being further arranged to receives the values as input values.

13. A method for the operation of a user interface for a magnetic resonance imager, involving the steps of
presenting information indicating the effects of the assignment of values to attributes used to influence the content of the visual presentation of an acquired magnetic resonance image,
choosing values for at least one such assignment based on the information presented,
assigning the chosen value to the attribute.
